(19) United States
(12) Plant Patent Application Publication
(10) Pub. No.: US 2008/0184432 P1
(43) Pub. Date: Jul. 31, 2008

(54) Cherry tree named 'PC8007-2'

(76) Inventor: Thomas K. Toyama, Eugene, OR (US)

Correspondence Address: STRATTON BALLEW, 213 S 12TH AVE, YAKIMA, WA, 98902, US

(21) Appl. No.: 11/895,279

(22) Filed: Aug. 22, 2007

Related U.S. Application Data
(60) Provisional application No. 60/839,924, filed on Aug. 23, 2006.

(51) Int. Cl. A01H 5/00

(52) U.S. Cl. PLT/297

(57) ABSTRACT

A new cultivar of sweet cherry tree named ‘PC8007-2’ is disclosed. The new cultivar is notable for its firm, flavorful fruit.

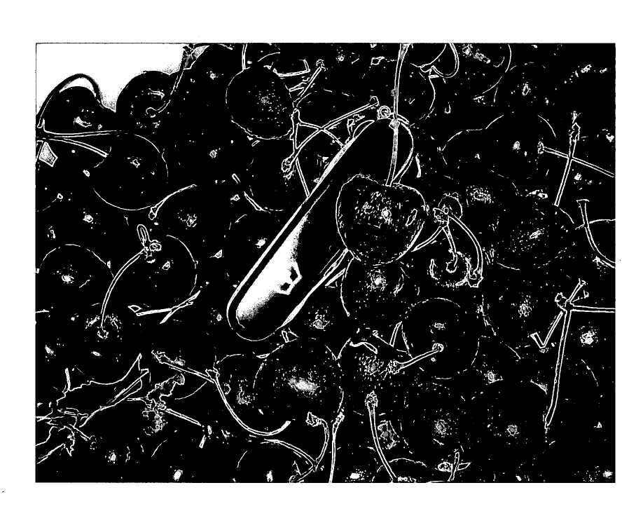

### PRIORITY CLAIM

This application claims priority of prior U.S. Provisional Application Ser. No. 60/839,924, filed Aug. 23, 2006.

### LATIN NAME OF THE GENUS AND SPECIES OF THE PLANT CLAIMED

Prunus avium

### VARIETY DENOMINATION

‘PC8007-2’

### BRIEF DESCRIPTION OF THE PHOTOGRAPHS

The photograph shows the fruit of the new cultivar.

### DETAILED BOTANICAL DESCRIPTION OF THE VARIETY

The present invention relates to a new variety of sweet cherry tree named ‘PC8007-2.’ ‘PC8007-2’ was developed at the Washington State University Irrigated Agriculture Research and Extension Center (I.A.R.E.C.) at Prosser, Washington. It was selected from among several seedlings that resulted from a controlled cross made in 1980 between ‘PC7144-7’ (female parent, not patented) and ‘PC7144-3’ (male parent, not patented), and has continued to be tested as ‘PC8007-2.’

Asexual reproduction of this new and distinct cultivar at test facilities near Prosser shows that its desirable characteristics come true to form and are established and transmitted through succeeding propagations by grafting.

The following is a detailed botanical description of ‘PC8007-2,’ based on observations made during the 2007 growing season in Prosser, Washington, of 25 year old trees grown on Mazzard rootstock. It should be understood that the botanical and analytical characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Semi-vigorous, productive; Height 5 m, spread 8.8 m; Trunk — diameter 57 cm at 15 cm above soil, bark rough, greyed-green 197A, trunk lenticel color greyed-orange 164B, trunk lenticels 5.5 mm, numerous; Branch — diameter 20.8 cm, length 4.6 m, crotch angle 48°, texture medium to rough, first year wood greyed-red 178B and greyed-brown 199D, second year wood greyed-red 182C and greyed-brown 199B, branch lenticels 9.6 mm, numerous, greyed-brown 199B.
Leaves: Size — length 13 mm, width 6 mm; Shape — elliptic, acute tip, rounded base, serrated margin; Color — upper surface green 141B, lower surface green 143A, midvein upper surface yellow-green 146C, midvein lower surface yellow-green 146C; Petiole — length 3.8 mm, diameter 1.6 mm; upper surface greyed-orange 166A, lower surface greyed-orange 166B; Glands — Quantity 2, reniform, length 2 mm, width 1 mm, color greyed-purple 185B.
Bloom time: 2 days later than ‘Bing’
Fruit: Size — wide plane diameter 29.0 mm, narrow plane diameter 24.8 mm, height 26.5 mm, weight 12.1 g; Shape — symmetrical; Suture — present, depth 0.4 mm; Stem — length 43.1 mm, diameter 1.2 mm, yellow-green 145A; Cavity — depth 1.5 mm, diameter 11.9 mm; Skin — smooth, thickness 0.5 mm, low tenacity, moderate tendency to crack, red-purple 59A; Flesh — red-purple 59A, smooth, medium juiciness, excellent eating quality; Stone — length 12.4 mm, width 7.5 mm, broad elliptic, tenacious, greyed-orange 164C.
Harvest: First picking June 18, last picking June 23, maturity date June 20 (2007 growing season, Prosser, Washington).

We claim:

1. A new and distinct cherry tree, substantially as shown and described herein.
